(19) United States
(12) Patent Application Publication
(10) Pub. No.: US 2013/0274845 A1
(43) Pub. Date: Oct. 17, 2013

(54) SYSTEMS, DEVICES AND METHODS FOR DISTAL FIXATION OF A MEDICAL DEVICE

(71) Applicant: NeuroAccess Technologies, Boston, MA (US)

(72) Inventors: Scott Kokones, Brookline, MA (US); Gregory Schulte, Minneapolis, MN (US); Shivanand Lad, Durham, NC (US); Vincent Owens, Hingham, MA (US); Andrew Firlik, Darien, CT (US)

(21) Appl. No.: 13/832,045

(22) Filed: Mar. 15, 2013

Related U.S. Application Data
(60) Provisional application No. 61/622,337, filed on Apr. 10, 2012.
(60) Provisional application No. 61/712,517, filed on Oct. 11, 2012.
(60) Provisional application No. 61/729,452, filed on Nov. 23, 2012.

(51) Int. Cl. A61N 1/05

(52) U.S. Cl. CPC A61N 1/0558
USPC 607/117

(57) ABSTRACT

Systems, devices and methods to anchor the distal portion of an implantable electrical lead using a coupling system to prevent or minimize lead migration. Magnetic-assisted anchoring systems are disclosed.

### CROSS-REFERENCE TO RELATED APPLICATIONS

The present application claims priority to U.S. Provisional Application No. 61/622,337 filed on Apr. 10, 2012 entitled: “Systems and Methods for Stimulating the Spinal Cord;” U.S. Provisional Application No. 61,712,517 filed on Oct. 11, 2012 entitled: “Lead Assemblies, Implantation Tools, Accessory Tools, and Systems and Methods of Using Same;” and U.S. Provisional Application No. 61/729,452 filed on Nov. 23, 2012 entitled: “Positioning Elements and Tools for Adding Positioning Elements to Leads and Catheters,” all of which are incorporated by reference herein in their entirety.

### TECHNICAL FIELD

The present invention relates to distal fixation of a medical device, such as an electrical lead.

### BACKGROUND

Electrical stimulation of the nervous system is used for a wide variety of medical conditions such as pain, depression, and Parkinson's Disease. One form of neurostimulation is spinal cord stimulation (SCS). SCS is mostly used for treating failed back syndrome, complex regional pain, and refractory pain due to ischemia. SCS has also been used for treating motor disorders, such as spasticity. One problem with current percutaneous SCS leads is migration from the implantation or target site. The lead or leads tend to move after implantation given their inherent in-line design. Surgical paddle-style leads tend to have less migration because they often scar in place due to their profile. Proximal fixation around the lead body of percutaneous stimulation leads is limited because such fixation can potentially damage the lead body and thus the electrical connections of the lead. Additionally, even when the lead is adequately secured proximal to the stimulation electrodes, there is a tendency for gravity to cause the stimulation electrodes to migrate caudally in the patient due to the fact that the patient spends considerable time standing or sitting upright.

In particular, existing anchors generally work in one of two ways. One way they are used is to first suture to the anchor to the lead body. This is done by first sliding the anchor over the lead body by threading the anchor over the proximal end of the lead and moving it toward the distal end of the lead. When in the desired place in the lead body, the anchor is sutured to the lead body. At times, this is either done too tightly creating stress on the conductors and potentially leading to fracture in the future if this portion is flexed. It can also be applied too loosely allowing the anchor to slip along the lead body reducing the strength of the anchor. The anchor is then sutured to the fascia. Another method is similar to the first where the anchor is threaded onto the lead and then makes a connection via a mechanical lock on the lead. In a twist lock anchor for example, a user twists the anchor reducing the inner diameter of the anchor causing the anchor to clamp down on the lead. The anchor is then sutured to the fascia similar to the other anchor. This regulates the force to give the ideal holding force without crushing the lead. The force a user can apply to the lead is still limited in this example because the conductors are in the lead body and are in danger of being fractured if the anchor is tightened too much.

As such, there is a need to securely fixate a lead, such as a SCS lead, at the implantation site without damaging the lead or lead accessories.

### SUMMARY

The present invention provides systems, devices and methods to anchor the distal portion of an implantable electrical lead or catheter using a coupling system to prevent or minimize lead migration. Although the below disclosure will be described primarily with respect to a magnetic-assisted anchoring system, other coupling systems can be used that include a coupler and a complimentary coupler that releasably attach to one another during use to form a removable coupling. Non-limiting examples of such couplers and complimentary couplers include male/female fasteners; hooks and loops; a suction device and suctionable device; and two cannulated shafts that couple to each via an interference fit.

In particular, the present invention provides different embodiments of devices, systems and methods that allow a user to anchor the distal portion of an implantable lead, preferably a SCS lead, or catheter instead of or in conjunction with the middle portion of the lead or catheter. In the case of a SCS lead, this can be accomplished by a) accessing a patient's epidural or intradural space in both a cephalad vertebral space and in a second caudal vertebral space; b) passing a device through an access opening to the cephalad vertebral space (referred to herein as a “cephalad access opening”) and passing another device through an access opening to the caudal vertebral space (referred to herein as a “caudal access opening”), wherein at least one of the devices comprises a magnet and the other device comprises a magnet or magnetic material; c) making a magnetic connection/forming a magnetic unit between the two devices within the epidural or intradural space and d) preferably using the cephalad access opening to provide an anchor point for a SCS lead or drug delivery catheter in conjunction with or as an alternative to mid-body lead anchoring systems. Although the present invention will primarily be described with respect to a lead, it is understood that the devices, systems and methods can also be used with a catheter and other implantable devices that require anchoring. Also, the present invention will primarily be described with respect to SCS, but can be used with other forms of therapy as well.

In an embodiment, the present invention provides a method of distally anchoring a lead. The lead comprises an electrical lead body having a proximal portion with a proximal end and a distal portion with a distal end. The lead also comprises an electrical conductor extending between the proximal portion and the distal portion. The lead further comprises an electrode array comprising an electrode in electrical communication with the electrical conductor, the electrode array located on the distal portion of the lead body. The lead includes a coupler located on the distal portion of the lead body. The method includes inserting the lead into the patient's body, positioning the lead at a target site of the patient's body and attaching the coupler of the lead to a location of the patient's body distal to the coupler.

In an embodiment, the present invention provides a lead anchoring system comprising a retrieval device and an anchoring device. The retrieval device comprises a distal tip comprising a coupler. The retrieval device also comprises a steering member extending proximally from the distal tip.

The anchoring device comprises a distal tip comprising a complimentary coupler. The anchoring device further comprises an anchor extending proximally from the distal tip. The couplers of the retrieval device and the anchoring device releasably attach to one another during use to form a removable coupling. The system further comprises an electrical lead comprising an elongate electrical lead body having a proximal portion and a distal portion. The lead further comprises an electrical conductor extending between the proximal portion and the distal portion. The lead also includes an electrode array comprising an electrode, and preferably a plurality of electrodes, in electrical communication with the electrical conductor. The electrode array is located on the distal portion of the lead body.

In an embodiment, the present invention provides a retrieval device comprising a distal tip and a steering member. The distal tip comprises a magnet or magnetic material (which may be referred to herein as a “magnetic tip” and encompasses a tip comprising a magnet or magnetic material). The steering member extends proximally from the tip.

In another embodiment, the present invention provides an anchoring device comprising a distal tip and an anchor, such as a filament. The distal tip comprises a magnet or magnetic material (which may be referred to herein as a “magnetic tip” and encompasses a tip comprising a magnet or magnetic material). The anchor extends proximally from the tip.

In another embodiment, the present invention provides a system comprising the retrieval device and the anchoring device described immediately above. The tips of the retrieval device and anchoring device are attracted to each other to form a magnetic connection or unit during use.

In another embodiment, the present invention provides a system comprising the retrieval device and anchoring device described immediately above and a lead. The lead can be a percutaneous lead or a paddle-style lead, for example. In an embodiment, the lead comprises an elongate electrical lead body having a proximal portion and a distal portion. An electrical conductor extends between the proximal portion and the distal portion. The electrical lead further comprises an electrode array comprising an electrode in electrical communication with the electrical conductor. The electrode array is located on the distal portion of the lead body.

In another embodiment, the present invention provides a method of anchoring a lead, such as in the epidural space or intradural space, of a patient's spinal canal. The method comprises forming a caudal access opening to the spinal canal and forming a cephalad access opening to the spinal canal. The method further comprises inserting the retrieval device described immediately above into one of the caudal access opening or the cephalad access opening. Additionally, the method comprises inserting an anchoring device described immediately above into the other of the caudal access opening or the cephalad access opening. The tips of the retrieval and anchoring device are attracted to each other such that they are capable of forming a magnetic connection/unit when they come into contact with each other. The retrieval device is urged into the spinal canal passage until the tip of the retrieval device contacts the tip of the anchoring device such that the two tips are magnetically joined to form a magnetic connection. This magnetic connection essentially connects the retrieval device to the anchoring device. The retrieval device can be urged into the spinal passage via the cephalad or caudal access opening, but preferably is introduced through the caudal access opening. The retrieval device is then retracted towards the access opening through which it was inserted until the anchor extends the length of the passage between the two access openings and extends out from the caudal and cephalad access openings. The method further comprises severing the anchor at a section close to the magnetic connection to remove the magnetic connection and the retrieval device's steering member. The severed end of the anchor is then attached to a lead, such as that described immediately above. Specifically, the severed end of the anchor is attached to the electrical lead's distal portion. The lead is advanced into the desired location within the spinal passage. The free end of the anchor is anchored to tissue, such as the fascia outside of the free end of the anchor. Thus the lead's distal portion is anchored in the spinal canal.

The present invention provides another embodiment of a retrieval device. This retrieval device comprises a tubular sleeve configured to attach to the distal portion of a lead. The sleeve comprises a distal tip comprising a magnet or magnetic material. The retrieval device further comprises a fastener located proximal to the distal magnetic tip that is configured to receive and fixate an anchor to the sleeve and, thus, to the distal portion of the lead.

The present invention also provides a system including the sleeve embodiment of a retrieval device immediately described above and an electrical lead to which the sleeve attaches. Alternatively or in addition, the system includes an anchoring device, such as the anchoring device described above.

The present invention also provides a method of using such a retrieval device. The method comprises forming a caudal access opening to the spinal canal and forming a cephalad access opening to the spinal canal. The method further comprises inserting an electrical lead having a proximal portion and a distal portion with the retrieval device's sleeve attached to the lead's distal portion into one of the caudal access opening or the cephalad access opening. Additionally, the method comprises inserting an anchoring device, such as described above, into the other of the caudal access opening or the cephalad access opening. The tips of the retrieval device and anchoring device are attracted to each other such that they are capable of forming a magnetic connection/unit when they come into contact with each other. The lead is urged into the spinal passage until the tip of the retrieval device's sleeve contacts the tip of the anchoring device such that the two tips are magnetically joined to form a magnetic connection. This magnetic connection essentially connects the lead (via the sleeve) to the anchoring device. The lead can be urged into the spinal passage via the cephalad or caudal access opening, but preferably is introduced through the caudal access opening. The lead with the retrieval device disposed thereon is then retracted towards the access opening through which it was inserted until the anchor extends the length of the passage between the two access openings and extends out from the caudal and cephalad access openings. The method further comprises severing the anchor at a section close to the magnetic connection to remove the magnetic connection. The severed end of the anchor is then attached to the fastener of the sleeve. The lead is advanced into the desired location within the spinal passage. The free end of the anchor is anchored to tissue, such as the fascia outside of the free end of the anchor. Thus, the lead's distal portion is anchored in the spinal canal.

The present invention provides another embodiment of an anchoring device. In this embodiment, the anchoring device comprises a proximal plug and a distal tip. The tip comprises a magnet or magnetic material. The device further comprises an anchor, such as a filament, that extends between the tip and plug. The proximal end of the anchor is attached to the plug and the distal end of the anchor is attached to the tip.

The present invention also provides a system comprising the above-described anchoring device and a retrieval device and lead. The retrieval device comprises a distal tip and optionally a steering member. The distal tip comprises a magnet or magnetic material. The steering member extends proximally from the distal tip. The tips of the retrieval device and anchoring device are attracted to each other to form a magnetic connection or unit during use. The lead comprises a proximal portion having a proximal end, a distal portion having a distal end, and a lumen extending between the proximal portion and the distal portion. The distal end of the lead is configured to prevent the plug from passing through a distal opening of the lumen (if a distal opening is present) and is configured to receive the magnetic tip of the anchoring device. For example, the lead can have a receptacle at its distal end to accept the magnetic tip of the anchoring device.

The present invention also provides a method of using the above-described anchoring device and retrieval device. The method comprises forming a caudal access opening to the spinal canal and forming a cephalad access opening to the spinal canal. The method further comprises inserting a lead having a proximal portion with a proximal end, a distal portion with a distal end, and a lumen extending between the proximal and distal portions into the spinal passage, either through the caudal access opening or the cephalad access opening, with the magnetic tip of the anchoring device seated on the distal end of the lead. Additionally, the method comprises inserting the retrieval device into the other of the caudal access opening or the cephalad access opening. The lead is urged into the spinal passage until the tip of the anchoring device contacts the tip of the retrieval device such that the two tips are magnetically joined to form a magnetic connection. This magnetic connection essentially connects the retrieval device to the anchoring device. The lead can be urged into the spinal passage via the cephalad or caudal access opening, but preferably is introduced through the caudal access opening. The retrieval device is then retracted towards the access opening through which it was inserted. As the retrieval device is retracted, the magnetic tip of the anchoring device is released from the distal end of the lead and the anchor withdraws from the central lumen of the lead until reaching the distal end of the lead, which is configured to prevent the plug of the anchoring device from exiting the lead's distal end. The method further comprises severing the anchor at a section close to the magnetic unit to remove the magnetic unit. The free end of the anchor in then attached to tissue, such as the fascia outside of the free end of the anchor. Thus, the lead's distal portion is anchored in the spinal canal.

In all the embodiments described herein, the anchor and/or steering member can be removably or non-removably attached to their respective tips (or plug) directly or indirectly so long as they do not disengage from their respective tips (or plug) during delivery through the spinal passage such that they cannot perform their intended function.

### BRIEF DESCRIPTION OF THE DRAWINGS

FIG. 1 is a schematic illustration of a lateral view of the vertebral column depicting a cephalad introduction device and a caudal introduction device according to an embodiment of the present invention.

FIG. 2 is a perspective view of a retrieval device according to an embodiment of the present invention.

FIG. 3 is a perspective view of an anchoring device according to an embodiment of the present invention.

FIG. 4 is a side view of an electrical lead according to an embodiment of the present invention.

FIG. 5 is a side view of a retrieval device attached to an anchor according to an embodiment of the present invention.

FIG. 6 is a perspective view of a retrieval device according to an embodiment of the present invention.

FIG. 6A is a cutaway view of the retrieval device of FIG. 6 according to an embodiment of the present invention.

FIG. 7 is a perspective view of a retrieval device according to an embodiment of the present invention.

FIG. 8 is a perspective view of an anchoring device in second configuration according to an embodiment of the present invention.

FIG. 9 is a perspective view of the anchoring device of FIG. 8 in a first configuration according to an embodiment of the present invention.

### DETAILED DESCRIPTION

The present invention provides systems, devices and methods for fixating a medical device at a target site or implantation site in a patient's body. In preferred embodiments, the patient is a mammal, such as a human being. In certain embodiments, the medical device delivers a therapy signal to a therapy site in the patients body. The therapy site can be the same site as the target site or can be adjacent to the target site. The medical device, for example, can be an electrical lead and/or a drug delivery catheter and the therapy signal can be an electrical signal and/or a chemical signal that provides a therapeutic effect to the patient. Although the present invention may be described with respect to SCS where the therapy site is the spinal cord and the target site is the epidural or intradural space of the spinal canal, the present invention can be used for other therapeutic purposes for other parts of a patient's body. For example, the present invention can be used for neuromodulation or other therapies of the brain; cranial nerves such as the vagus nerve; peripheral nerves such as spinal nerves including the occipital nerve and sacral nerve; and other regions of the nervous system, both the central and peripheral nervous system. The systems, devices, and methods can be used for deep brain leads, gastric leads, vagus nerve leads, peripheral nerve leads including occipital nerve and sacral nerve leads, drug delivery catheters, cardiac catheters, and cardiac stimulation leads.

The disclosure herein may refer to electrical or neural “stimulation” or “modulation.” Such terms include both inhibition or activation of electrical activity in and/or around the therapy site. Further, as used herein with respect to a described component, feature, or element, the terms “a,” “an,” and “the” include at least one or more of the described component, feature, or element unless otherwise indicated.

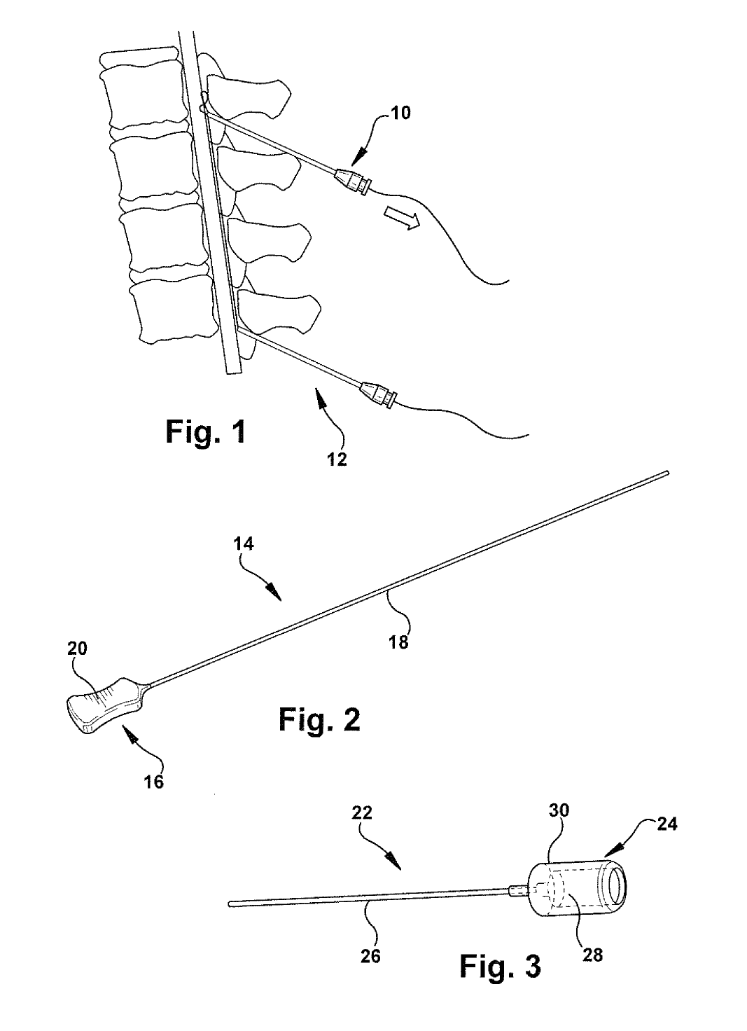

In the case of SCS, the spinal cord may be accessed through various different methods. A preferred access method is a spinal needle to minimize invasiveness. Furthermore, a touhy-style needle allows devices, such as a lead, catheter, anchoring device, or retrieval device, being passed through the needle to bend at an angle upon exit from the needle lumen. This allows advancement of the device along the axis of the spinal cord. Different introduction devices may be used at the different entry sites. For example, the introduction device may be a guidewire, or a guidewire combined with a needle. Different introducers including plastic or expandable introducers can be passed to access the epidural or intradural space using an over-the-wire approach. In embodiments of the present invention, two access openings are used—one at each end of the spinal passage being accessed. Specifically, in the case of SCS stimulation, introduction devices create a cephalad access opening and a caudal access opening. FIG. 1 illustrates two introduction devices 10 and 12 (e.g. needles). Introduction device 10 (referred to as a “cephalad needle”) is used to pass or retrieve a cephaladic device (described in more detail herein). The cephalad access opening should be close to the desired distal end of the lead location (preferably the next caudal vertebral space), since this will also be the anchor location in most cases. Introduction device 12 (referred to herein as a “caudal needle”) is used to pass a caudal device (referred to in more detail herein) into the spinal passage and to pass the lead into place. It may be desirable to insert the cephalad needle with a retrograde approach so that devices that are passed through the cephalad and caudal needles are directed at each other upon insertion.

Referring to FIG. 2, in an embodiment, the present invention provides a retrieval device 14. Retrieval device 14 comprises a distal tip 16 and a steering member 18. Distal tip 16 comprises a magnet or magnetic material 20. Steering member 18 extends proximally from distal tip 16. In a preferred embodiment, the retrieval device is a caudal device. By “caudal device” is meant a device that is inserted through a caudal needle into the caudal access opening created by the caudal needle. Referring to FIG. 3, the present invention also provides an anchoring device 22 comprising a distal tip 24 and an anchor 26. Distal tip 24 comprises a magnet or magnetic material 28. Anchor 26 extends proximally from distal tip 24. The anchor may be described herein as a suturable filament but other types of anchors can be used. In a preferred embodiment, the anchoring device is a cephalad device. By “cephalad device” is meant a device that is inserted through a cephalad needle into the cephalad access opening created by the cephalad needle. The tips 16 and 24 of the retrieval and anchoring devices are attracted to each other to form a magnetic connection or unit in the spinal passage. As shown in FIG. 3, in certain embodiments, the magnetic tip of either or both the devices is encased in plastic 30 or other suitable material.

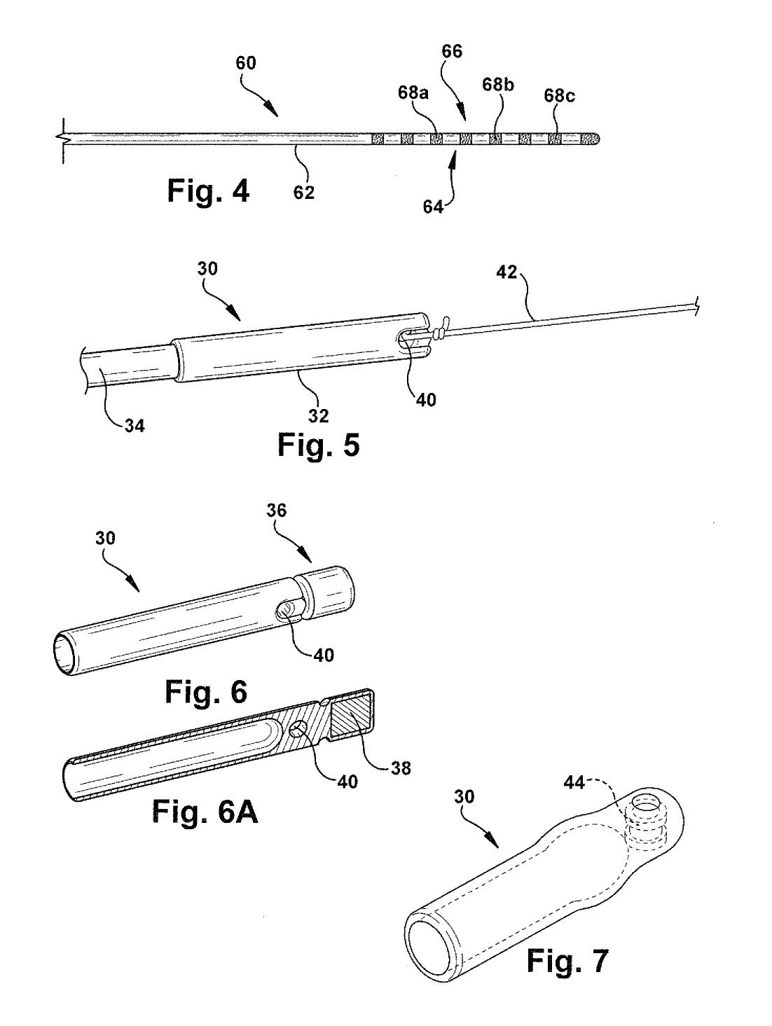

Referring to FIG. 4, the present invention also provides an electrical lead 60. Lead 60 comprises an elongate electrical lead body 62 having a proximal portion (not shown) and a distal portion 64. An electrical conductor (not shown) extends between the proximal portion and the distal portion. The electrical lead further comprises an electrode array 66 comprising an electrode 68, and preferably a plurality of electrodes, in electrical communication with the electrical conductor. The electrode array is located on distal portion 64 of lead body 62.

In a preferred embodiment of a method of the present invention, a retrieval device is inserted through the caudal access opening and advanced in a cephaladic direction in the spinal passage. An anchoring device (as shown in FIG. 3, for example) is inserted through the cephalad access opening and the two devices are magnetically joined to form a magnetic connection/unit with fluoroscopic assistance.

In this embodiment and certain other embodiments, the filament should be minimally the length of the passage between the access openings so that it always may be accessed outside of the spinal canal. In a preferred embodiment, the magnetic connection (described above) is withdrawn through the caudal access opening by pulling back the caudal device, but the filament remains extended through the entire spinal passage between the two access openings and extends out from the cephalad access opening (as well as the caudal access opening). The filament is cut at a point close to the magnetic connection at the caudal access opening to remove the magnetic connection and steering member of the caudal device. The filament is attached to the SCS lead's distal portion. The lead is advanced into place with or without a stylet, or can be pulled into place using the filament's free end at the cephalad access opening or a combination of both. The filament is anchored to the fascia, for example, outside of the cephalad access opening after the appropriate amount of filament slack has been removed.

The steering member of the retrieval device can be constructed similar to an electrical stimulation lead such as a polyurethane or silicone tube, since a physician is comfortable guiding this construction to the desired location. It can also be a metal coil which has desirable stiffness and torquability for steering. It may simply comprise a stylet or guidewire type construction. A bendable stylet may be used within the steering member to help guide it such as is used in an SCS lead. A stylet is bendable in the sense that a physician or other user can manipulate the stylet to create a bent shape in the stylet and then manipulate the stylet again to create an un-bent shape without using other instrumentation to accomplish this and without damaging the integrity (i.e. breaking) of the stylet such that it no longer performs its intended function. The anchoring device can have a steering member as well and this steering member can be of similar construction and materials as the steering member of the retrieval device.

In another embodiment, the present invention provides systems, methods and devices for distal fixation of a lead that can be used with existing SCS leads. This is advantageous because the anchoring method can be used without modifying currently available percutaneous stimulation leads. Referring to FIG. 5, in particular, in an embodiment, the present invention provides a retrieval device 30 comprising a tubular sleeve 32 configured to attach to the distal end of a lead 34. Referring to FIGS. 6 and 6A (which is a cross section of the retrieval device of FIG. 6), sleeve 32 comprises a distal tip 36 comprising a magnet or magnetic material 38. Sleeve 32 further comprises a fastener 40 located proximal to the distal magnetic tip 36 that is configured to receive and fixate an anchor 42, such as a filament, to lead 34 during the implantation procedure. Fastener 40 can be an eyelet or through-hole through which a filament is passed and knotted to secure a filament to the sleeve and thus to the lead during use (as shown in FIG. 5). The eyelet or through-hole can be reinforced with a metal grommet or ring 44 as shown in FIG. 7. The fastener can be any suitable fixation system that secures the filament to the lead, such as a groove, eyelet, through-hole, set screw, or other component.

A system of the present invention can provide the retrieval device as described immediately above and an anchoring device as described above and/or a lead as described above.

The sleeve can be an elastic tube that fits tightly over the lead outer diameter (OD) and is held in place via an interference fit. The sleeve can comprise any suitable elastic material such as, but not limited to silicone, polyurethane or a blend of silicone and polyurethane. An appropriate inner diameter (ID) for the sleeve may be 0.025-0.055″ since this is smaller than the OD of the lead body. The sleeve can be placed onto the lead by rolling the sleeve into a donut shape and then unrolling it onto the lead tip. It can also be expanded with a tool and slid over the lead tip before the tool is removed. Attachment of the sleeve is not limited to elastic interference fit. Additional methods include suturing a sleeve over the OD of the lead, crimping a malleable material such as platinum or MP35N over the lead's OD, using a medical adhesive to bond the sleeve to the lead, or mechanical lock onto the lead such as a setscrew onto a distal electrode OD.

An exemplary method of using the above described retrieval device will now be described. The method comprises forming a caudal access opening to the spinal canal and forming a cephalad access opening to the spinal canal. The method further comprises inserting the lead with the sleeve attached to the distal end of the lead into one of the caudal access opening or the cephalad access opening. Additionally, the method comprises inserting an anchoring device into the other of the caudal access opening or the cephalad access opening. The anchoring device comprises a distal tip comprising a magnet or magnetic material. The anchoring device further comprises an anchor, such as a filament, extending from the distal tip. The tips of the sleeve and anchoring device are attracted to each other such that they are capable of forming a magnetic connection/unit when they come into contact with each other. The lead is urged into the spinal passage until the tip of the sleeve contacts the tip of the anchoring device such that the two tips are magnetically joined to form a magnetic unit. This magnetic unit essentially connects the lead (via the sleeve) to the device. The lead can be urged into the spinal passage via the cephalad or caudal access opening, but preferably is introduced through the caudal access opening. The lead is then retracted towards the access opening through which it was inserted until the anchor extends the length of the spinal passage and extends out from the caudal and cephalad access openings. The method further comprises severing the anchor at a section close to the magnetic unit to remove the magnetic unit. To the retrieval device's fastener, the physician attaches the anchor end extending from the opening through which the retrieval device was retracted. The lead is advanced into the desired location within the spinal passage. The other end of the anchor is anchored to tissue, such as the fascia outside of the other end of the anchor. Thus the lead's distal portion is anchored in the spinal canal.

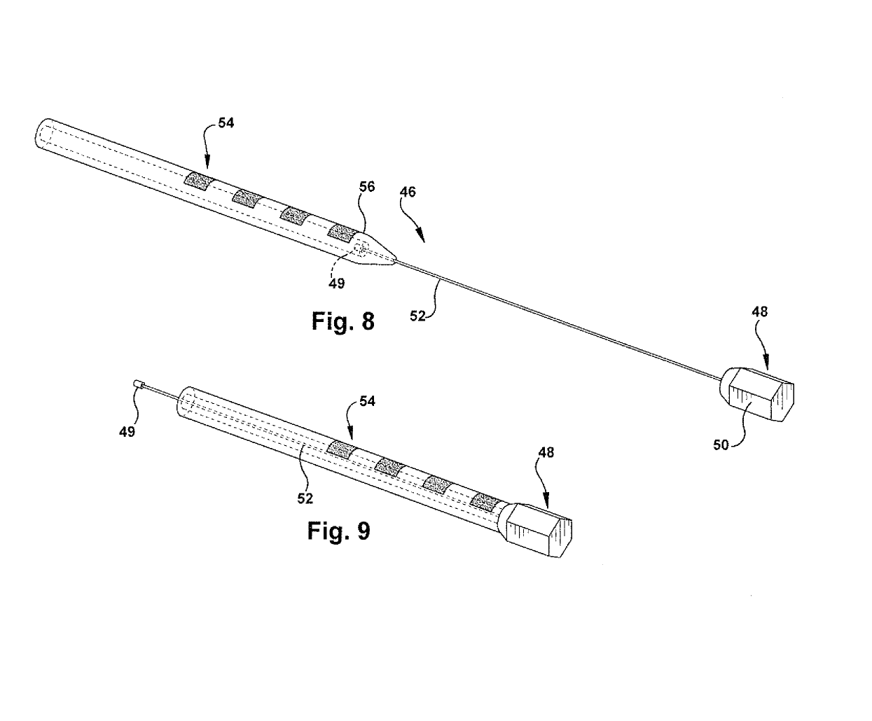

Referring to FIGS. 8 and 9, in another embodiment, the present invention provides an anchoring device 46 comprising a distal tip 48 and a proximal plug 49. Distal tip 48 comprises a magnet or magnetic material 50. Anchoring device 46 further comprises an anchor 52 extending between and in communication with the distal tip 48 and proximal plug 49. In particular, the anchor's proximal end can be attached to plug 49, which can be a filament bead, and the anchor's distal end can be attached to tip 48. In certain embodiments, the plug is integral with the anchor and is not a separate component, in the case of a filament and a filament bead as shown in FIGS. 8 and 9. The anchoring device can be used in conjunction with a lead 54 as shown in FIGS. 8 and 9. As such, the present invention also provides a system comprising a lead and the above-described anchoring device. Tip 48 of anchoring device 46 is configured to releaseably fit on the tip 56 of lead 54 as shown in FIG. 9. There are various mechanisms by which this releasable fixation can be accomplished, such as interference fit, withdrawal of a thin sheath on the outside diameter of the lead, or mechanical locking features that are released with a tool such a stylet.

The lead can be specifically designed to accommodate the device. In an embodiment, the lead has a lumen, such as a central lumen for example, that extends the length of the lead body. The lumen allows the anchor to pass through the lead body until reaching the distal end of the lead, which can be tapered or have different configuration such that the plug does not exit the distal end of the lead. The lumen of the lead body can also accommodate a guiding stylet.

In another embodiment, the present invention provides a system comprising the above-described anchoring device and a retrieval device, such as retrieval devices describe above. Alternatively or in addition, the system can include a lead that can properly accommodate the magnetic tip of the anchoring device.

According to a method of the present invention, the anchoring device first assumes a first configuration as shown in FIG. 9. This is the configuration of the device prior to implanting the device and before retrieval of the device in the spinal passage. In this configuration, the magnetic tip is releaseably attached to the lead's tip and the filament extends through the lead body's lumen. The magnetic tip is seated at the distal tip of the lead until the lead is positioned at a desired location in the spinal passage. Specifically, the lead is urged through the spinal passage, preferably through the caudal access opening, until reaching the desired location. Once at such location, a retrieval device comprising a distal tip comprising a magnet or magnetic material that is attracted to the magnetic tip of the anchoring device contacts the magnetic tip of the anchoring device forming a magnetic connection/unit. The retrieval device is then retracted towards the access opening through which it was inserted. As the retrieval device is retracted, the magnetic tip of the anchoring device is released from the tip of the lead and the anchor withdraws from the central lumen of the lead until reaching the distal end of the lead, which is configured to prevent the plug of the anchoring device from exiting the lead's distal end. The method further comprises severing the retrieval device at a section close to the magnetic unit to remove the magnetic unit. The physician then anchors the free end of the anchor to tissue, such as the fascia outside of the distal end of the anchor.

In a preferred embodiment, the retrieval device is a cephalad device. This preferred embodiment differs from the above-described preferred embodiment as the magnetic unit is retrieved through the cephalad access opening. The plug also acts as a seal to fluid ingress. The plug may be fabricated from a soft silicone material. The plug can also be configured as a grommet so it seals itself after retracting the stylet. The lead lumen may be tapered to the distal end to allow the filament to pass freely but restrict the plug and stylet from poking through the distal tip. Additionally, a specific stylet with a tapered distal end may be used to release the magnet from its seating at the end of the lead. The lead tip may be shaped to accommodate the magnet with an interference fit to avoid the magnet from coming loose while retrieval device through the introducer needle and spinal passage. The magnet may have machined featured or may be overmolded with plastic features that assist in holding the magnet in place while it is being passed.

With respect to any of the embodiments described herein, the magnet may be protected with a material such as, for example, parylene, polyurethane, silicone, PEEK, ULTEM, polycarbonate or other materials, to make it more biocompatible. The magnet may be encased in material such as titanium to facilitate biocompatibility or additional mechanical features. In any of the embodiments described above, the magnet may have cylindrical shape or other shape. The magnetic material may be any suitable magnetic material, such as a ferromagnetic material. In a preferred embodiment, the magnetic material is biocompatible or enclosed in a casing fabricated from a biocompatible material.

With respect to the anchor, in a preferred embodiment, the anchor is a suture filament. One end of the filament is attached to the lead (or an intermediate device such as a sleeve that is fixed to the lead as described above). The other end can be fixed to a needle that may be used to secure the filament to tissue. The other end can also have nothing attached to it as described above. The filament used to attach to the magnet or magnetic material may be a suture wire, or a biocompatible material including but not limited to polyethylene, nylon, polyester, silk, polypropylene, other standard suture materials, or a metallic cable such as a BSW wire.

As stated above, although the examples have detailed methods for spinal cord stimulation, all of the above methods can be used for placement of other stimulation leads such as peripheral stimulation leads, occipital stimulation leads, vagus nerve stimulation and stimulation of the sacral nerve. Alternatively, this method could be used for implantation of drug delivery catheters, cardiac catheters and cardiac stimulation leads.

The detailed distal anchoring methods and devices may also be used in combination with traditional anchoring methods. When used with traditional anchoring systems that are fixed to the lead body proximal to the electrodes, a loop or strain relief may be created distal to the lead body anchor during implantation in order to create slack.

In another embodiment, the present invention provides a method of distally anchoring an electrical lead. The lead comprises an electrical lead body having a proximal portion with a proximal end and a distal portion with a distal end. The lead further comprises an electrical conductor extending between the proximal portion and the distal portion. An electrode array comprising an electrode is located on the distal portion of the lead body and is in electrical communication with the electrical conductor. The lead can be a percutaneous lead (e.g. a cylindrical lead with the electrode array disposed about the distal portion of the lead body) or a paddle-style (e.g. where the distal portion of the lead is a paddle). The lead further includes a first coupler located on the distal portion of the lead body. The method includes inserting the lead into the patient's body, positioning the lead at a target site of the patients body, and attaching the coupler of the lead to a location of the patient's body distal to the coupler. For example, the coupler can be a through-hole and attaching the coupler comprises fastening one end of a suture to the through-hole and attaching the opposing end of the suture to a location of the patient's body distal to the through-hole. The lead can also include a second coupler located between the proximal end and the distal end of the lead body and/or a third coupler located on the proximal portion of the lead body. In certain embodiments, the target site is the spinal canal of the patient's body and the location of the patient's body is fascia outside of the spinal canal.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A lead anchoring system comprising:
a retrieval device comprising:
a distal tip comprising a coupler; and
a steering member extending proximally from the distal tip; and
an anchoring device comprising:
a distal tip comprising a complimentary coupler; and
an anchor extending proximally from the distal tip,
wherein the couplers of the retrieval device and the anchoring device releasably attach to one another during use to form a removable coupling; and
an electrical lead comprising:
an elongate electrical lead body having a proximal portion and a distal portion;
an electrical conductor extending between the proximal portion and the distal portion; and
an electrode array comprising an electrode in electrical communication with the electrical conductor, the electrode array located on the distal portion of the lead body.

2. A lead anchoring system comprising:
a retrieval device comprising:
a distal tip comprising a magnet or magnetic material; and
a steering member extending proximally from the distal tip;
an anchoring device comprising:
a distal tip comprising a magnet or magnetic material; and
an anchor extending proximally from the distal tip,
wherein the distal tips of the retrieval device and the anchoring device are attracted to each other to form a magnetic connection during use; and
an electrical lead comprising:
an elongate electrical lead body having a proximal portion and a distal portion;
an electrical conductor extending between the proximal portion and the distal portion; and
an electrode array comprising an electrode in electrical communication with the electrical conductor, the electrode array located on the distal portion of the lead body.

3. The system of claim 2, wherein the magnet or the magnetic material of the distal tip of the retrieval device is encased or overmolded with a non-magnetic material.

4. The system of claim 2, wherein the magnet or the magnetic material of the distal tip of the anchoring device is encased or overmolded with a non-magnetic material.

5. The system of claim 2, wherein the anchor is a suture.

6. The system of claim 2, wherein the steering member is an elongated shaft.

7. The system of claim 2, wherein the steering member is a coil.

8. The system of claim 2, wherein the steering member is a bendable stylet.

9. A method of anchoring a lead in a patient's spinal canal comprising:
forming a caudal access opening to a patient's spinal canal;
forming a cephalad access opening to the patient's spinal canal;
inserting a retrieval device into one of the caudal access opening or the cephalad access opening, the retrieval device comprising:
a distal tip comprising a magnet or magnetic material; and
a steering member extending proximally from the distal tip;
inserting an anchoring device into the other of the caudal access opening or the cephalad access opening, the anchoring device comprising:
a distal tip comprising a magnet or magnetic material; and
an anchor extending proximally from the distal tip,
wherein the distal tips of the retrieval and anchoring devices are attracted to each other such that they are capable of forming a magnetic connection when they come into contact with each other;
urging the retrieval device into a spinal passage between the caudal and cephalad openings of the spinal canal until the distal tip of the retrieval device contacts the distal tip of the anchoring device such that the two distal tips are magnetically joined to form a magnetic connection;
retracting the retrieval device towards the access opening through which it was inserted until the anchor extends the length of the spinal passage between the caudal and cephalad access openings;
severing the anchor at a section close to the magnetic connection;
removing the magnetic connection and the steering member of the retrieval device;
attaching the severed end of the anchor to a lead's distal end;
advancing the lead into the desired location within the spinal passage; and
attaching the free end of the anchor to a site in the patients body.

10. The method of claim 9, wherein the retrieval device is inserted into the caudal access opening and urged in a cephaladic direction in the spinal passage.

11. The method of claim 10, wherein the anchoring device is inserted into the cephalad access opening.

12. The method of claim 9, wherein the magnetic connection is withdrawal through the caudal access opening by pulling back the retrieval device.

13. The method of claim 12, wherein the anchor is severed at a section close to the magnetic connection at the caudal access opening to remove the magnetic connection and the steering member of the retrieval device.

14. The method of claim 11, wherein the anchor is attached to the fascia outside of the cephalad access opening.

15. The method of claim 9, wherein the anchor is a suture,

16. A lead anchoring system comprising:
a retrieval device comprising:
a tubular sleeve having a distal tip comprising a magnet or magnetic material; and
a fastener located proximal to the distal tip;
an anchoring device comprising:
a distal tip comprising a magnet or magnetic material; and
an anchor extending proximally from the distal tip,
wherein the distal tips of the retrieval device and the anchoring device are attracted to each other to form a magnetic connection during use; and
an electrical lead comprising:
an elongate electrical lead body having a proximal portion and a distal portion;
an electrical conductor extending between the proximal portion and the distal portion; and
an electrode array comprising an electrode in electrical communication with the electrical conductor, the electrode array located on the distal portion of the lead body.

17. The system of claim 17, wherein the fastener is a through-hole.

18. The system of claim 17, wherein the through-hole is reinforced.

19. A method of anchoring a lead in a patient's spinal canal comprising:
forming a caudal access opening to a patient's spinal canal;
forming a cephalad access opening to the patient's spinal canal;
inserting a lead having a retrieval device disposed about the distal end thereof into one of the caudal access opening or the cephalad access opening, the retrieval device comprising:
a tubular sleeve having a distal tip comprising a magnet or magnetic material; and
a fastener located proximal to the distal tip;
inserting an anchoring device into the other of the caudal access opening or the cephalad access opening, the anchoring device comprising:
a distal tip comprising a magnet or magnetic material; and
an anchor extending proximally from the distal tip,
wherein the distal tips of the retrieval and anchoring devices are attracted to each other such that they are capable of forming a magnetic connection when they come into contact with each other;
urging the lead with the retrieval device attached thereto into a spinal passage between the caudal and cephalad openings of the spinal canal until the distal tip of the retrieval device contacts the distal tip of the anchoring device such that the two distal tips are magnetically joined to form a magnetic connection;
retracting the lead towards the access opening through which it was inserted until the anchor extends the length of the spinal passage between the caudal and cephalad access openings;
severing the anchor at a section close to the magnetic connection;
removing the magnetic connection;
attaching the severed end of the anchor to the retrieval device's fastener;
advancing the lead into the desired location within the spinal passage; and
attaching the free end of the anchor to a site in the patient's body.

20. The method of claim 19, wherein the lead is inserted into the caudal access opening and urged in a cephaladic direction in the spinal passage.

21. The method of claim 20, wherein the anchoring device is inserted into the cephalad access opening.

22. The method of claim 19, wherein the anchor is severed at a section close to the magnetic connection at the caudal access opening to remove the magnetic connection of the retrieval device.

23. The method of claim 19, wherein the anchor is attached to the fascia outside of the cephalad access opening.

24. A lead anchoring system comprising:
an anchoring device comprising:
a distal tip comprising a magnet or magnetic material;
a proximal plug; and
an anchor extending between and in communication with the distal end and proximal plug;
and
an electrical lead comprising:
an elongate electrical lead body having a proximal portion and a distal portion with a distal end, the distal tip of the anchoring device configured to releasably fit on the distal end of the lead body;
an electrical conductor extending between the proximal portion and the distal portion; and
an electrode array comprising an electrode in electrical communication with the electrical conductor, the electrode array located on the distal portion of the lead body.

25. The anchoring device of claim 24, wherein the proximal plug is a filament bead.

26. The anchoring device of claim 24, wherein the anchor is a filament.

27. The anchoring device of claim 24, wherein the anchor's proximal end is connected to the proximal plug.

28. The anchoring device of claim 24, wherein the anchor's distal end is connected to the distal tip.

29. The system of claim 24, wherein the distal end of the lead is tapered to prevent exit of the proximal plug.

30. The system of claim 24, wherein the lead comprises a lumen extending between the proximal portion and the distal portion, the lumen configured to allow passage of the anchor.

31. A method of anchoring a lead in a patient's spinal canal comprising:
providing an anchoring device comprising:
a distal tip comprising a magnet or magnetic material;
a proximal plug; and
an anchor extending between and in communication with the distal tip and proximal plug;
providing a lead having a proximal end, a tapered distal end configured to prevent exit of the proximal plug, and a lumen extending therebetween configured to allow passage of the anchor;
attaching the distal tip of the anchoring device to the distal end of the lead and allowing the anchor to extend through the lead's lumen;
inserting a retrieval device into one of a caudal access opening or a cephalad access opening, the retrieval device having a distal tip comprising a magnet or magnetic material;
inserting the lead with the anchoring device attached thereto into the other of a caudal access opening or a cephalad access opening, the distal tip of the retrieval device and the distal tip of the anchoring device attracted to each other such that they are capable of forming a magnetic connection when they come into contact with each other;
urging the lead with the anchoring device attached thereto into a spinal passage between the caudal and cephalad openings of the spinal canal until reaching the desired implantation location;
allowing the distal tip of the retrieval device to contact the distal tip of the anchoring device such that the two distal tips are magnetically joined to form a magnetic connection;
retracting the retrieval device towards the access opening through which it was inserted;
allowing the distal tip of the anchoring device to release from the distal end of the lead and the anchor to withdraw from the lumen of the lead until the proximal plug contacts the tapered distal end of the lead;
severing the anchor device at a section close to the magnetic connection;
removing the magnetic connection; and
attaching the free end of the anchor to a site in the patient's body.

32. The method of claim 31, wherein the retrieval device is inserted in the cephalad access opening.

33. A method of anchoring a lead comprising:
providing an electrical lead comprising:
an electrical lead body having a proximal portion with a proximal end and a distal portion with a distal end;
an electrical conductor extending between the proximal portion and the distal portion;
an electrode array comprising an electrode in electrical communication with the electrical conductor, the electrode array located on the distal portion of the lead body; and
a first coupler located on the distal portion of the lead body;
inserting the lead into the patient's body;
positioning the lead at a target site of the patient's body;
attaching the coupler of the lead to a location of the patient's body distal to the coupler.

34. The method of claim 33, wherein the electrical lead is a cylindrical lead having an elongated lead body and the electrode array is disposed about the distal portion the lead body.

35. The method of claim 33, wherein the electrical lead is inserted percutaneously.

36. The method of claim 33, wherein the distal portion of the lead comprises a paddle.

37. The method of claim 33, further comprising a second coupler located between the proximal end and the distal end of the lead body.

38. The method of claim 33, further comprising a third coupler located on the proximal portion of the lead body,

39. The method of claim 33, wherein the target site is the spinal canal of the patient's body.

40. The method of claim 39, wherein the location of the patient's body is fascia outside of the spinal canal.

41. The method of claim 33, wherein the coupler is a through-hole and attaching the coupler comprises fastening one end of a suture to the through-hole and attaching the opposing end of the suture to a location of the patient's body distal to the through-hole.
